(19) United States
(12) Patent Application Publication
(10) Pub. No.: US 2026/0212963 A1
(43) Pub. Date: Jul. 23, 2026

(54) A COMPUTER IMPLEMENTED METHOD AND AN APPARATUS FOR NON-INVASIVE AND EXTRA-CORPORALLY DETERMINING THE ARTERIAL CONCENTRATION OF A RADIOPHARMACEUTICAL, IN PARTICULAR A RADIOTRACER IN AN ORGAN OF AN ANIMAL AND/OR HUMAN BODY

(71) Applicants: Universiteit Maastricht, Maastricht (NL); Academisch Ziekenhuis Maastricht, Maastricht (NL)

(72) Inventor: Roel Wierts, Maastricht (NL)

(21) Appl. No.: 19/143,977

(22) PCT Filed: Dec. 13, 2023
(86) PCT No.: PCT/EP2023/085609

§ 371 Date: Jun. 27, 2025

(30) Foreign Application Priority Data
Dec. 30, 2022 (EP) 22217263.7

(51) Int. Cl. G16C 20/20; G16H 50/30

(52) U.S. Cl. CPC G16C 20/20; G16H 50/30

(57) ABSTRACT

The present disclosure relates to a technique to non-invasively and extra-corporeally measure the arterial concentration of a radiotracer administered to animal and/or human bodies or subjects with a radiation detector. In particular a computer implemented method for non-invasive and extra-corporally determining the arterial concentration of a radiopharmaceutical, for example a radiotracer, in an organ of an animal and/or human body is proposed, the computer implemented method comprising the steps of: i) receiving, from the moment of administration of a certain amount of the radiopharmaceutical in the animal and/or human body, by one or more radiation sensors positioned near or on a measurement location of an organ electromagnetic radiation emitted by the radio-pharmaceutical over time; ii) converting the received electromagnetic radiation in a time-sequence of radiation signals; iii) generating one or more time-frequency representations of the radiation signals by time-frequency transformation, and iv) analyzing the time-frequency representations for determining the arterial concentration of the radiopharmaceutical.

### TECHNICAL FIELD

The present disclosure relates to a technique to non-invasively and extra-corporeally measure the arterial concentration of a radiopharmaceutical, in particular a radiotracer administered to animal and/or human bodies or subjects with a radiation detector/sensor.

### BACKGROUND OF THE DISCLOSURE

Current clinical quantification of (¹⁸F-FDG) PET images is based on the assessment of radiotracer uptake at a specific time point after the administration of the radiotracer using the standardized uptake value (SUV). Although the SUV is the most commonly used PET metric in clinical practice, it suffers from a number of important shortcomings. Besides the radiotracer uptake time, the measured SUV depends on the total amount cumulative radiotracer concentration in the arterial blood over time. In the prior art, it has been described that these shortcoming may result in erroneous conclusions with respect to therapy response in oncologic patients using the SUV.

Alternatively, if the time-dependent arterial radiotracer concentration is known, the SUV can be corrected for the radiotracer availability and the true metabolic activity of tissue can be determined. This will have an added value on the clinical value of (18F-FDG) PET imaging. Besides a correction for the SUV, the arterial radiotracer concentration over time is also required to perform pharmacokinetic modelling which is typically used in research application for various (new) PET radiotracers.

A known measuring technique for arterial radiotracer concentration assessment requires the placement of an arterial line in patients/subjects. Arterial line placement is invasive, time consuming and not without risk. Therefore, this technique is not used in clinical practice. Another known technique is known as image-derived input function (IDIF), which technique requires lengthy dynamic PET acquisitions of typically 30-60 minutes after the administration of the radiotracer in a blood vessel. The arterial radiotracer concentration is then determined from the reconstructed PET images at various time points. As this technique requires lengthy PET scans, the number of patients that can be scanned is greatly reduced. This technique is therefore not applicable to current clinical PET scanning in which short static (whole-body) PET scans are performed after a specific uptake time.

Alternatively, another known approach is based on population studies, wherein the general shape of the ¹⁸F-FDG radiotracer concentration over time was determined for constructing a population-based input function. Using one or more venous blood samples, this population-based input function can be scaled for individual patients. However, this technique suffers from inter-and intra-patient variations of the time-dependent arterial radiotracer concentration.

Accordingly, it is a goal of the present disclosure to provide an improved method and apparatus for non-invasively and extra-corporally determining the arterial concentration of a radiopharmaceutical in an organ of an animal and/or human body. The radiopharmaceutical used can be a radiotracer, more in particular of a positron emission tomography (PET) radiotracer.

### SUMMARY OF THE DISCLOSURE

A first example of the disclosure proposes a computer implemented method for non-invasive and extra-corporally determining the arterial concentration of a radiopharmaceutical, for example a radiotracer, in an organ of an animal and/or human body. The computer implemented method comprising the steps of:
i) receiving, from the moment of administration of a certain amount of the radiopharmaceutical in the animal and/or human body, by one or more radiation sensors positioned near or on a measurement location of an organ electromagnetic radiation emitted by the radiopharmaceutical over time;
ii) converting the received electromagnetic radiation in a time-sequence of radiation signals;
iii) generating one or more time-frequency representations of the radiation signals by time-frequency transformation, and
iv) analyzing the time-frequency representations for determining the arterial concentration of the radiopharmaceutical.

By measuring the time-dependent radiopharmaceutical or radiotracer concentration directly following the administration of a radiopharmaceutical (radiotracer) in a blood vessel of the animal or human body and without the need to apply invasive arterial blood sampling or lengthy/costly data acquisitions, e.g. PET acquisitions, a direct and accurate correction of the SUV is achieved. Accordingly, this direct and accurate correction of the SUV has an added value on the clinical value of e.g. (¹⁸F-FDG) PET imaging. Besides a correction for the SUV, the radiopharmaceutical concentration over time in the arterial blood perfusing the organ is also required to perform pharmacokinetic modeling which is typically used in research application for various (new) radiotracers, such as PET radiotracers.

In an advantageous example of the disclosure, the time-frequency transformation is selected from the group exemplified by but not limited to short-time Fourier transform, wavelet transform, filter bank, or discrete cosine transform. This results in an analysis in the time-frequency domain, with a calculation of the correct measurement of the time-dependent radiotracer concentration in the organ, e.g. the heart or artery. Although the concentration is not measured directly from the signals acquired with the radiation sensors, the acquired signals can be used to obtain the actual measurement, for example on the basis of a PET/SPECT scan or on the basis of a venous blood sample acquired at a later stage.

In a further detail of the disclosure, step i) comprises the step of receiving the electromagnetic radiation over time using two radiation sensors positioned at opposite sides of the measurement location of the organ. Herewith a more accurate measurement can be obtained by using the so-called coincidence radiation emission principle.

To further improve the accuracy of the measurement of the time-dependent radiotracer concentration, step i) further comprises the step of collimating the electromagnetic radiation emitted by the radiopharmaceutical prior to the reception by the one or more radiation sensors. Herewith radiation safety is guaranteed, and only electromagnetic radiation emanating from the measurement location of the organ is being detected and analyzed for the measurement.

The disclosure also pertains to an apparatus for the non-invasive and extra-corporally determination of the concentration of a radiopharmaceutical, in particular a radiotracer administered in an blood vessel of an animal and/or human body. Preferably the apparatus is structured to implement the computer-implemented method. The apparatus may comprise in a preferred example one or more radiation sensors to be positioned near or on a measurement location of an organ of the animal and/or human body and configured to receive electromagnetic radiation emitted by the radiopharmaceutical over time. The apparatus also may comprise a conversion unit configured to convert the received electromagnetic radiation over time in a time-sequence of radiation signals as well as at least one processing unit configured to generate one or multiple time-frequency representations of the time-sequence of radiation signals through time-frequency transformation, and for determining the concentration of the radiopharmaceutical.

With this configuration a non-invasive and extra-corporal measuring device is obtained for determining the arterial concentration of a radiopharmaceutical in an organ (e.g. heart or artery) of an animal and/or human body. Accordingly, due to the non-invasive characteristic, the apparatus can be implemented under in a less strict medical circumstances with readily simple sensing equipment, limiting the burden to the animal and/or human subject.

In particular, an output unit is used configured to output the concentration of the radiopharmaceutical being determined, for example via a display.

For obtaining the measurement results fast and efficient in terms of computation time, the time-frequency transformation is selected from the group exemplified by but not limited to short-time Fourier transform, wavelet transform, filter bank, or discrete cosine transform.

In a preferred example of the apparatus according to the disclosure, two radiation sensors are positioned at opposite sides of the measurement location of the organ. In this particular example, the apparatus may comprise a coincidence processing unit structured to detect the simultaneous detection of electromagnetic radiation by the two radiation sensors positioned at opposite sides of the measurement location of the organ. Herewith a more accurate measurement is possible of the concentration of the radiopharmaceutical and a direct and accurate correction of the SUV is achieved. Accordingly, his direct and accurate correction of the SUV has an added value on the clinical value of e.g. (¹⁸F-FDG) PET imaging. Besides a correction for the SUV, the radiopharmaceutical concentration over time is also required to perform pharmacokinetic modeling which is typically used in research application for various (new) radiopharmaceutical, e.g. PET radiotracers.

In a further example, the apparatus may comprise a housing for accommodating at least the one or more radiation sensors, as well as radiation collimating means positioned around each of the one or more radiation sensors. This facilitates an easier handling or manipulation of the apparatus according to the disclosure, as well as this configuration improves radiation safety and only electromagnetic radiation emanating from the measurement location of the organ is being detected and analyzed for the measurement.

In a preferred example, the apparatus is formed as a wearable device, for example structured for wearing around a limb of the animal and/or human body and on or near the measurement location of the organ. Accordingly, due to each non-invasive characteristic, the apparatus can be implemented under in a less strict medical circumstances with readily simple sensing equipment, limiting the burden to the animal and/or human subjects.

For example, in a beneficial example allowing a simple, yet direct implementation during (¹⁸F-FDG) PET images acquisition, the apparatus is structured to positioned near, at or incorporated in a hospital chair, a hospital bed or patient table.

In other advantageous embodiments, the method of the present disclosure can be embodied in a computer program or product, which computer program or product comprises computer-coded instructions which, when the computer program or product program is executed by a computer, such as a laptop or computer, cause the computer to carry out the steps of the computer implemented method disclosed herein.

In a particular embodiment, a computer-readable storage medium is proposed comprising computer-coded instructions stored therein, which computer-coded instructions, when executed by a computer, cause the computer to carry out the steps of the computer implemented method disclosed in this application. Such computer-readable storage medium can be a (solid-state) hard drive, or a USB drive, or a (digital) optical disc.

### BRIEF DESCRIPTION OF THE DRAWINGS

The disclosure will now be discussed with reference to the drawings, which show in:

FIG. 1 a schematic example of an apparatus according to the disclosure implementing an example of a method according to the disclosure;

FIG. 2 a more detailed example of an apparatus according to the disclosure implementing an example of a method according to the disclosure;

FIG. 3 a detailed example of a radiation sensor for use in an apparatus according to the disclosure;

FIGS. 4A and 4B further examples of an apparatus according to the disclosure implementing an example of a method according to the disclosure;

FIGS. 5A and 5B details of signals acquired and processed in an example of a method according to the disclosure.

### DETAILED DESCRIPTION OF THE DISCLOSURE

For a proper understanding of the disclosure, in the detailed description below corresponding elements or parts of the disclosure will be denoted with identical reference numerals in the drawings.

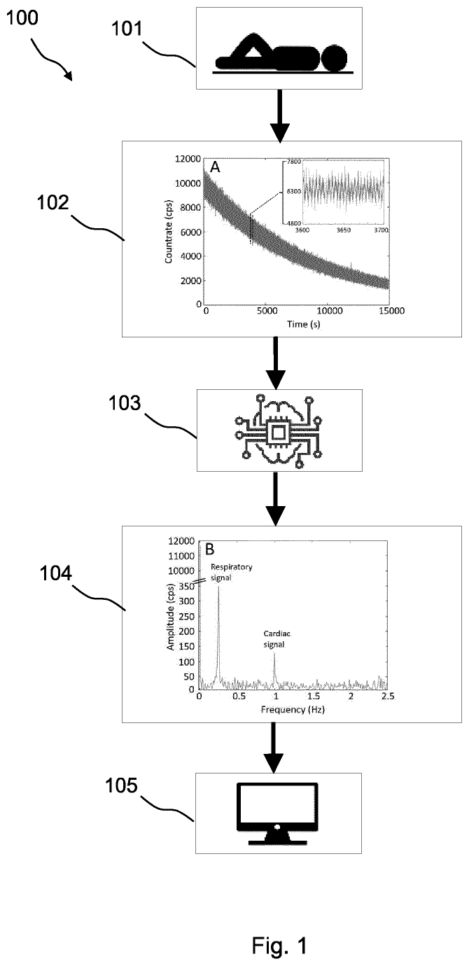

FIG. 1 depict a first schematic example of a computer implemented method for non-invasive and extra-corporally determining the arterial concentration of a radiopharmaceutical in an organ of an animal and/or human body according to the disclosure. It is noted that any radiopharmaceutical can be used, but in particular a radiotracer. The method and apparatus are equally applicable with radiopharmaceuticals to be used in radionuclide therapy, as with the acquisition of similar measuring signals in radionuclide therapy a better prediction of the effectiveness of the therapy can be made.

The non-invasive computer implemented method proposes an improved alternative compared to the present day clinical quantifications of e.g. (¹⁸F-FDG) PET images, which present day clinical quantifications are based on the assessment of radiotracer uptake at a specific time point after the administration of the radiotracer using the standardized uptake value (SUV). As outlined in the introduction of this application, although the SUV is the most commonly used PET metric in clinical practice, it suffers from a number of important shortcomings. Besides the radiotracer uptake time, the measured SUV depends on the total amount cumulative radiotracer concentration in the arterial blood over time. In the prior art, it has been described that these shortcoming may result in erroneous conclusions with respect to therapy response in oncologic patients using the SUV.

Accordingly, In FIG. 1 an improved method for non-invasively and extra-corporally determining the arterial concentration of a radiopharmaceutical, in particular a radiotracer, more in particular the arterial concentration of a positron emission tomography (PET) radiotracer in an organ of an animal and/or human body is explained below.

The computer implemented method is preferably implemented in an apparatus for the non-invasive and extra-corporally determination of the arterial concentration of a radiopharmaceutical, in particular a radiotracer injected in an blood vessel of an animal and/or human body. In FIG. 1 the apparatus is denoted with reference numeral 100 and will be outlined in more detail further in the detailed description.

In FIG. 1, the computer implemented method is structured along four steps. The first step, denoted by the box with reference numeral 101, concerns a patient which is positioned or lying in a treatment area 101 on a hospital chair, a hospital bed or patient table, which may be part of the apparatus 100. In the patient, which may be an animal or a human, a certain amount of radiopharmaceutical is administered, e.g. injected in the blood stream, e.g. in a blood vessel. The radiopharmaceutical can be any type of radiopharmaceutical, e.g. selected from the group of radiotracers, and in particular can be ¹⁸F-FDG also known as [¹⁸F]Fluorodeoxyglucose, or fluorodeoxyglucose F 18. Fluorodeoxyglucose ([¹⁸F]FDG, 2-[¹⁸F]FDG or FDG) is a radiopharmaceutical, specifically a radiotracer, used in the medical imaging modality positron emission tomography. However, other types of known radiopharmaceuticals can also be used with the method and apparatus according to the disclosure.

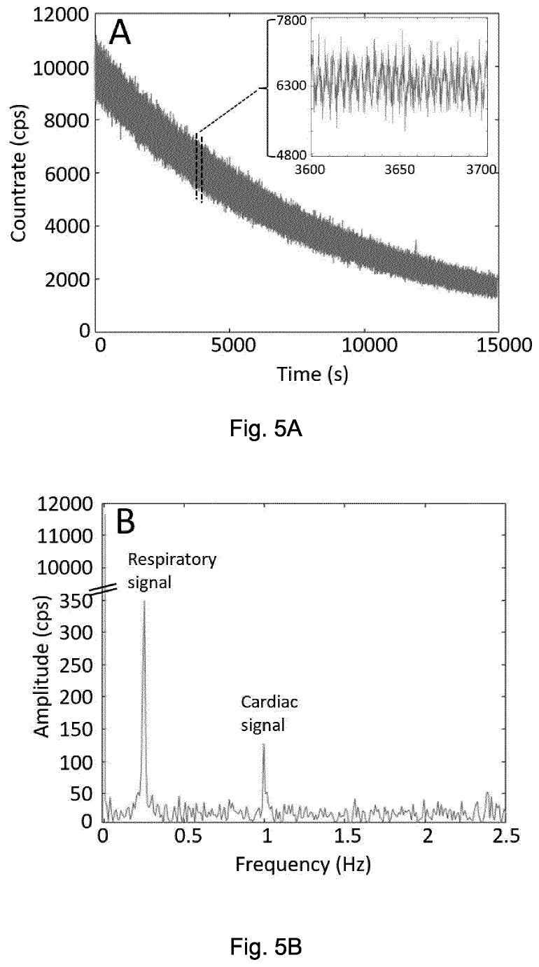

The computer implemented method according to the disclosure in particular focuses in its first step i), that, immediately or from the moment of administration of the certain amount of the radiopharmaceutical the blood stream in the animal and/or human body, electromagnetic radiation emitted by the radiopharmaceutical being injected is received over time by one or more radiation sensors which are positioned near or on a measurement location of an organ of interest. This is depicted in box 102 of FIG. 1 and depicted in more detail in FIG. 5B, both showing the electromagnetic radiation emitted by the radiopharmaceutical being received (detected) over time.

The organ can be the heart of the animal or human, or any other part of its body which part is well perfused with blood. The organ of interest can be a limb such as a hand or foot or part of a limb such as an ankle or wrist, as long as the one or more radiation sensors can be positioned closely to a blood vessel, but most preferably to blood artery, e.g. of the wrist or ankle arteries, which perfuses the respective organ of interest.

In a next step ii) of computer implemented method according to the disclosure the received electromagnetic radiation are converted in a time-sequence of radiation signals, as depicted schematically by box 103 and box 104. Preferably, the time-frequency transformation is short-time Fourier transform, but it should be noted that the time-frequency transformation as implemented by the computer implemented method according to the disclosure is not limited to Fourier transform. Also other time-frequency transformations can be used, e.g. selected from the group exemplified by but not limited to wavelet transform, filter bank, or discrete cosine transform.

Accordingly, implementing a time-frequency transformation on the time-sequence of radiation signals results in an analysis in the time-frequency domain, and this step iv) of analyzing the time-frequency representations leads to an accurate determination of the concentration of the radiopharmaceutical and calculation of the correct measurement of the time-dependent radiotracer concentration in the organ, e.g. the heart or artery as shown by the peaks in box 104 and depicted in more detail in FIG. 5B.

Although the concentration is not measured directly from the signals acquired with the radiation sensors, the acquired signals can be used to obtain the actual measurement, for example on the basis of a PET/SPECT scan or on the basis of a venous blood sample acquired at a later stage.

By measuring the time-dependent radiopharmaceutical or radiotracer concentration directly following the administration of a radiopharmaceutical (radiotracer) in a blood vessel of the animal or human body and without the need to apply invasive arterial blood sampling or lengthy/costly data acquisitions, e.g. PET acquisitions, a direct and accurate correction of the SUV is achieved. Accordingly, this direct and accurate correction of the SUV has an added value on the clinical value of e.g. (¹⁸F-FDG) PET imaging. Besides a correction for the SUV, the radiopharmaceutical concentration over time in the organ is also required to perform pharmacokinetic modeling which is typically used in research application for various (new) radiotracers, such as PET radiotracers. When the radiopharmaceutical or radiotracer undergoes radioactive decay, it forms a positron, and the positron combines with an electron to produce electromagnetic radiation in the form of gamma rays. These gamma rays are detected by means of a radiation sensor.

As outlined, the one or more radiation sensors are to be positioned closely to a blood vessel or blood artery which perfuses the organ of interest. For example, in the event that the organ of interest is a hand or foot, a more accurate measurement can be obtained by using the so-called coincidence radiation emission principle. According to this principle, step i) is further detailed as the step may comprise the step of receiving the electromagnetic radiation over time using two or more radiation sensors operating in coincidence detection mode which are positioned at opposite sides of the measurement location of the organ of interest.

A further improvement of the accuracy of the measurement of the time-dependent radiotracer concentration can be achieved, when step i) further comprises the step of collimating the electromagnetic radiation emitted by the radiopharmaceutical prior to the reception by the one or more radiation sensors. Herewith radiation safety is guaranteed, as well as measurement accuracy as only electromagnetic radiation emanating from the measurement location of the organ is being detected and analyzed for the measurement. It is noted that the step of collimating the electromagnetic radiation is not necessarily applicable, when the so-called coincidence radiation emission principle is implemented, as the event of two coinciding radiation emissions is accurate measurement in itself.

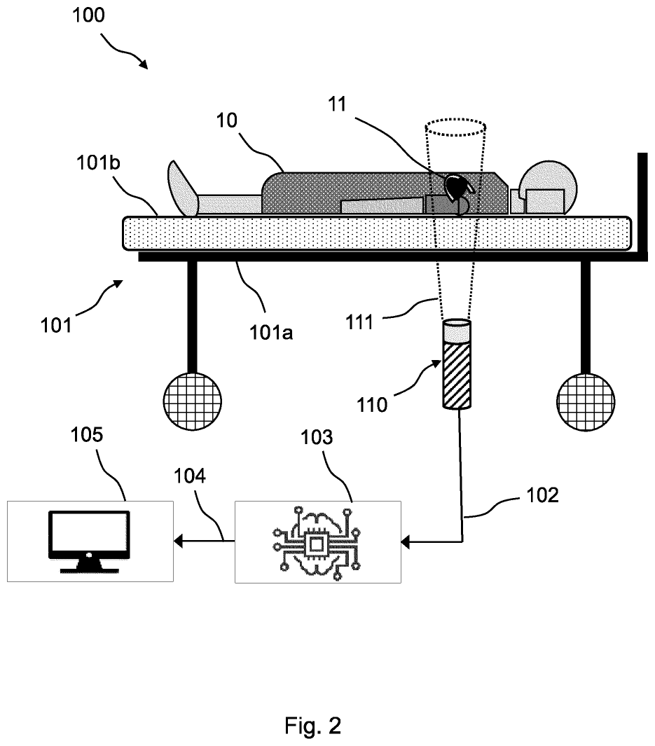

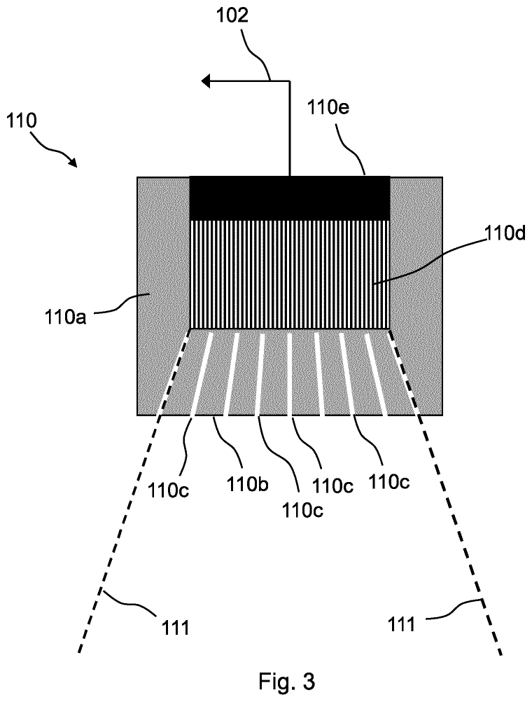

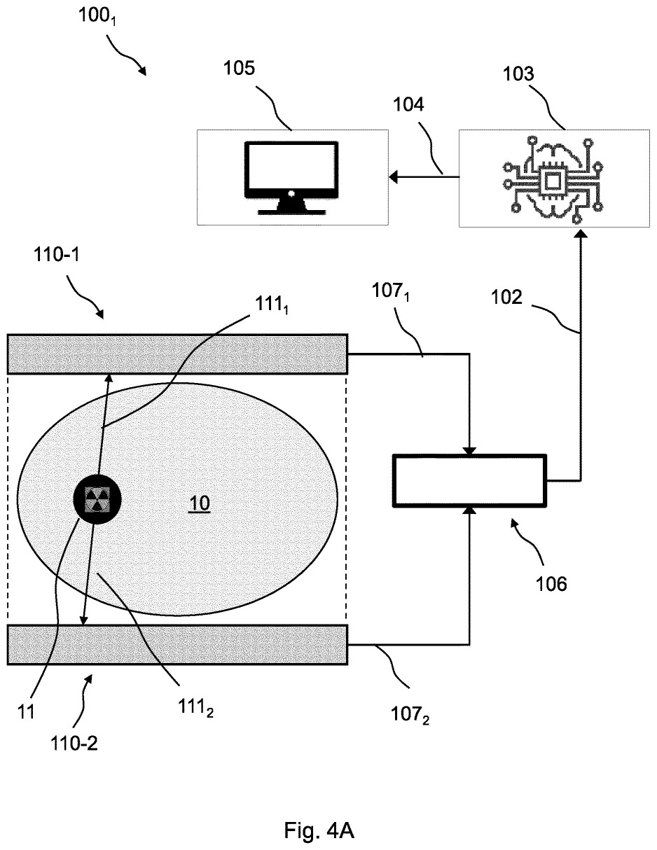

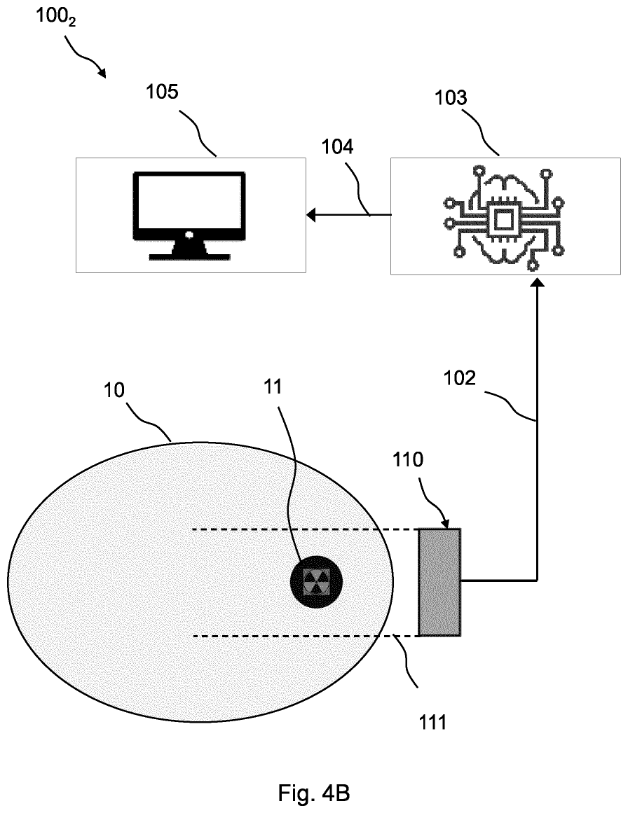

An example of an apparatus for the non-invasive and extra-corporally determination of the arterial concentration of a radiopharmaceutical, in particular a radiotracer administered in an blood vessel of an animal and/or human body implementing the computer-implemented method according to the disclosure is shown in FIG. 2, with details of the apparatus shown in FIG. 3 and FIGS. 4A and 4B.

The apparatus is denoted with reference numeral 100 and may be implemented or incorporated in a treatment area 101, such a surgery room or treatment room. Reference numeral 101a denotes a hospital chair, a hospital bed or patient table, e.g. a patient scanner table. In the example of the patient table 101a it may comprises a mattress 101b on which a subject to be examined can be positioned. The subject can be an animal or a human 10. For the sake of explanation, the apparatus 100 according to the disclosure will be explained as being implemented on a human patient 10.

In the patient 10 (animal or a human) is a certain amount of radiopharmaceutical administered e.g. by means of injection in the blood stream, e.g. in a blood vessel. One or more radiation sensors 110 are positioned near or on a measurement location of an organ of interest 11 of the animal and/or human body 10. In this particular application of the apparatus according to the disclosure, the apparatus 100 implements one radiation sensor 110, which is oriented towards an organ of interest, here the heart 11. It is essential for both the method and the apparatus according to the disclosure, that the organ of interest 11 can be any body part of the animal and/or human body 10, which body part is well perfused with blood. The organ of interest can be a limb such as a hand or foot, as long as the one or more radiation sensors can be positioned closely to preferably a blood artery which perfuses the organ of interest 11. In particular, the method and apparatus according to the disclosure can be used on or near the heart or on/around an ankle or a wrist, with the one or more radiation sensors measuring the electromagnetic radiation emitted by the radiopharmaceutical passing through the heart artery or ankle arteries or wrist arteries.

The one radiation sensor 110 as used in the example of FIG. 2 has an outer surface 110b (see FIG. 3) facing the organ of interest 11 and is configured to receive electromagnetic radiation 111 over time, which radiation 111 is emitted by the injected radiopharmaceutical during the perfusing of the organ of interest 11.

Referring to FIG. 3, an example of such radiation sensor 110 is shown. The radiation sensor 110 may comprise a radiation sensing area or radiation sensing element 110d which is accommodated in a housing 110a. The housing 110a incorporates the outer, sensing surface 110b as well as radiation collimating means 110c positioned around the radiation sensing element 110d. The outer, sensing surface 110b serves to receive the electromagnetic radiation 111 and the radiation collimating means 110c serve to collimate and direct only electromagnetic radiation 111 emitted by the radiopharmaceutical in the blood stream, which passes or perfuses the measurement location of the organ of interest 11 towards the radiation sensing element 110d. The radiation collimating means 110c may be structured as small bores in the sensing surface 110b of the housing 110a, which bores 110c may point in a converging manner towards the radiation sensing element 110d.

The housing 110a can be manufacturing of a radiation shielding material or contain radiation shielding elements in order to prevent stray radiation not originating from the measurement location of the organ of interest 11 from being detected by the radiation sensing element 110d. Accordingly, the radiation collimating means/bores 110c and optionally the radiation shielded housing 110a ensure that only electromagnetic radiation 111 emanating from the measurement location of the organ of interest 11 will be detected for the measurement, thus improving the accuracy thereof.

The radiation sensor 110 thus constructed from a housing 110a incorporating the necessary components for detecting the electromagnetic radiation 111 for the measurement, facilitates a more easy handling or manipulation of the apparatus according to the disclosure, as well as this configuration improves radiation safety.

The apparatus 100 may incorporate several radiation sensors 110-1, 110-2, 110-3, . . . , 110-n, with n being an positive number from one, two or more. An embodiment of an apparatus implementing one radiation sensor 110 is depicted in FIG. 2, with its detailed configuration shown in FIG. 3 and a schematic depiction of such single sensor application being depicted in FIG. 4B. In this example 100₂ of the apparatus according to the disclosure (shown in FIGS. 2 and 4B), one radiation sensor 110 is implemented, which receives via the sensing surface 110b as well as via radiation collimating means 110c (see FIG. 3) electromagnetic radiation 111 emitted by the radiopharmaceutical in the blood stream, which passes or perfuses the measurement location of the organ of interest 11.

The collimated electromagnetic radiation 111 is received by the radiation sensing element 110d in which the detected radiation can be converted in a corresponding electronic signal by means of a conversion unit 110e. For example, if the radiation sensor is constructed as a scintillation detector, the absorbed radiation energy will first be converted by the scintillation crystal (re. reference numeral 110d) into a (visible) light signal, after which the light signal will then be converted into an electrical signal by the photomultiplier (or APD, SiPM) (re. reference numeral 110e). In an example, wherein the radiation sensor is a semiconductor, a direct conversion into electrical signal may take place.

In this particular example the conversion unit 110e is part of the radiation sensor 110. However it should be noted that the conversion unit 110e can be implemented as a separate component 110e of the apparatus 100, not necessarily being part of the radiation sensor 110.

The conversion unit 110e is capable of converting in a known manner the electromagnetic radiation 111 being detected over time by the radiation sensing element 110d in a suitable time-dependent electronic signal 102. Such time-dependent electronic signal 102 can have the form as depicted in FIG. 5A and has a typical periodical shape due to the pulsating character of the arterial blood stream passing/perfusing the measurement location of the organ of interest 11 due to the heart beat rate of the animal/human 10.

Reference numeral 103 denotes another relevant component of the apparatus 100 according to the disclosure, and is incorporated as at least one processing unit 103 configured to generate one or multiple time-frequency representations of the time-sequence of radiation signals 102 through time-frequency transformation.

An example of a time-frequency representation of the time-sequence of radiation signals 102 is denoted with reference numeral 104 and shown for example in more detail in FIG. 5B. The time-frequency transformation of the time-sequence of radiation signals 102 decomposes the time-sequence of radiation signals 102 into its frequency components, which are represented by the output of the transform as a function of frequency, as shown in FIG. 5B. In this explanatory example of the time-frequency transformation of FIG. 5B, two frequency peaks are decomposed from the time-sequence of radiation signals 102. One frequency peak may correspond with the respiratory rhythm of the subject 10, whereas one other frequency peak may correspond with the pulsation of the blood circulation of the subject 10.

The frequency output of the transformation can be processed and will ultimately provide the concentration or amount of the radiopharmaceutical in the blood stream which has passed and perfused the measurement location of the organ of interest 11.

Accordingly, a non-invasive and extra-corporal measurement is obtained of the arterial concentration/amount of a radiopharmaceutical in an organ (e.g. heart or artery) of the animal and/or human body 10. Accordingly, due to the non-invasive characteristic, the apparatus 100 can be implemented under in a less strict medical circumstances with readily simple sensing equipment, limiting the burden to the animal and/or human subject 10.

For practical use, an output unit 105 may be used, which is configured to output the arterial concentration of the radiopharmaceutical thus determined, for example via a monitor or display. Also other output means can be implemented, such as print unit for a proper reporting on paper.

For obtaining the measurement results fast and efficient in terms of computation time, the time-frequency transformation as performed by the at least processing unit 103 is preferably short-time Fourier transform. However, as stated above, the time-frequency transformation as implemented by the at least processing unit 103 is not limited to Fourier transform. Also other time-frequency transformations can be used, e.g. selected from the group exemplified by but not limited to wavelet transform, filter bank, or discrete cosine transform.

Furthermore, although in the examples shown in the Figures only one processing unit 103 is used, it should be noted that the apparatus 100 according to the disclosure may implement several processing units 103, denoted with 103-1, 103-2, . . . 103-n, etc. Each processing unit 103-n can be allocated to each radiation sensor 110-n, thus being used for performing the time-frequency transformation of the time-sequence of radiation signals 102 generated by the respective radiation sensor 110-n.

Another example of the apparatus 100₁ according to the disclosure is shown in FIG. 4A. This example 100₁ implement two radiation sensors 110-1 and 110-2, which are positioned at opposite sides of the measurement location of the organ of interest 11. For example, the organ of interest 11 may a wrist artery or ankle artery and accordingly the apparatus 100₁ may be formed as a wearable device, for example structured for wearing around a limb (wrist or ankle) of the animal and/or human body 10 and on or near the measurement location of the organ of interest 11 (for example a wrist artery or ankle artery). Also in this particular example 100₁ of the apparatus of the disclosure, due to each non-invasive characteristic, the apparatus 100₁ can be implemented under in a less strict medical circumstances with readily simple sensing equipment, limiting the burden to the animal and/or human subjects 10.

As shown in FIG. 4A, two radiation sensors 110-1 and 110-2, which are positioned at opposite sides of the measurement location of the organ of interest 11. The apparatus 100₁ furthermore implements a coincidence processing unit 106, which is structured to detect the simultaneous detection of electromagnetic radiation 111₁ by the first radiation sensor 110-1 as well as electromagnetic radiation 111₂ by the other radiation sensor 110-2 positioned opposite of the first radiation sensor 110-1 and positioned at either side of the measurement location of the organ 11.

In particular, the coincidence processing unit 106 can be implemented when a radiopharmaceutical or radiotracer is used, which radiotracer undergoes radioactive decay and forms a positron, which positron in turn annihilates with an electron, thereby releasing two anti-colinear high-energy photons (gamma rays). These two high-energy photons 111-1 and 111-2 of each 511 keV propagate in opposite directions and can thus be simultaneously detected by the two radiation sensors 110-1 and 110-2, each oriented at opposite sides of the organ 11.

The simultaneous detection by the two radiation sensors 110-1 and 110-2 results in so-called trigger signals 107-1 and 107-2, which trigger signals are processed by the coincidence processing unit 106, and ultimately results in a time-sequence of radiation signals 102. Thus with this configuration a more accurate measurement is possible of the concentration (amount) of the radiopharmaceutical in the blood stream perfusing the organ of interest 11 and a direct and accurate correction of the SUV is achieved. Accordingly, the direct and accurate correction of the SUV has an added value on the clinical value of e.g. (¹⁸F-FDG) PET imaging. Besides a correction for the SUV, the arterial radiopharmaceutical concentration over time is also required to perform pharmacokinetic modeling which is typically used in research application for various (new) radiopharmaceutical, e.g. PET radiotracers.

Likewise in this particular example, both radiation sensors 110-1 and 110-2 can be implemented without the radiation collimating means 110c as used in the single radiation sensor example of the apparatus 100₁ of FIG. 4B (and FIG. 3).

With the implementation of coincidence detection, it will also be possible of using the positions of the simultaneously detected high-energy photons 111-1 and 111-2 on the detectors to identify, based on the early measurements immediately after administering the radiopharmaceutical, which connecting lines between the opposite detectors (the so-called line-of-response) pass through or intersect with the (arterial) blood vessels, which perfuse the organ 11. As these connecting lines do not change during the measurement, the data thus acquired could be used to look only at those line-of-responses and eliminate the rest. Particularly for measurements at a later time moment, when the arterial concentration may be very low and the background signal from the surrounding tissue may be high, this is an important method to further reduce the background signal.

In other advantageous embodiments, the method of the present disclosure can be embodied in a computer program or product, which computer program or product comprises computer-coded instructions which, when the computer program or product program is executed by a computer, such as a laptop or computer, cause the computer to carry out steps of the computer implemented method disclosed herein.

In a particular embodiment, a computer-readable storage medium is proposed comprising computer-coded instructions stored therein, which computer-coded instructions, when executed by a computer, cause the computer to carry out steps of the computer implemented method disclosed in this application. Such computer-readable storage medium can be a (solid-state) hard drive, or a USB drive, or a (digital) optical disc.

The example 100₁ of the apparatus according to the disclosure as shown in FIG. 4A implements two radiation sensors 110-1 and 110-2 and can be set up as a so-called synchronous arterial PET scanner (synchroPET scanning device),

Contrary to the known PET systems which derive the arterial radiotracer concentration in an image-derived way, both examples 100₁ (FIGS. 4A) and 100₂ (FIG. 4B) of the apparatus according to the disclosure may be constructed as a miniature arterial PET scanning system capable of assessing the arterial radiotracer concentration in the blood stream from the periodic variation in the measured radiation signal originating from cardioventricular contractions or arterial vessel pulsation.

This technique allows for the use of a relatively standard low-cost radiation detector without the need for creating 3D tomographic images of the radiotracer distribution in the patient's body. It also allows for the accurate and personalized measurement of an arterial radiotracer concentration in a non-invasive way, without the need for invasive arterial blood sampling, image-derived techniques or population-based generic input functions.

### LIST OF REFERENCE NUMERALS USED

10 animal body, human body, patient
11 measurement location, point/region of interest, organ
100, 100₁, 100₂ non-invasive, extra-corporal determination apparatus (1^{st}, 2^{nd}, 3^{rd} example of the disclosure)
101 treatment area
101a hospital bed
101b mattress
102 time-sequence of radiation signals
103 processing unit
104 time-frequency representations of the radiation signals
105 output unit
106 coincidence processing unit
107₁, 107₂ simultaneous detected radiation signals
110, 110-n radiation sensor(s)
110a housing/radiation shielding
110b entrance surface of housing
110c radiation collimating means
110d sensing surface, sensing area
110e conversion unit
111, 111₁, 111₂ electromagnetic radiation

1. A computer implemented method for non-invasive and extra-corporally determining the arterial concentration of a radiopharmaceutical, for example a radiotracer, in an organ of an animal and/or human body, the computer implemented method comprising the steps of:
i) receiving, from the moment of administration of a certain amount of the radiopharmaceutical in the animal and/or human body, by one or more radiation sensors positioned near or on a measurement location of an organ electromagnetic radiation emitted by the radiopharmaceutical over time;
ii) converting the received electromagnetic radiation in a time-sequence of radiation signals;
iii) generating one or more time-frequency representations of the radiation signals by time-frequency transformation, and
iv) analyzing the time-frequency representations for determining the arterial concentration of the radiopharmaceutical.

2. The computer implemented method of claim 1, wherein the time-frequency transformation is selected from the group exemplified by but not limited to short-time Fourier transform, wavelet transform, filter bank, or discrete cosine transform.

3. The computer implemented method of claim 1, wherein step i) comprises the step of receiving the electromagnetic radiation over time using two radiation sensors positioned at opposite sides of the measurement location of the artery.

4. The computer implemented method of claim 1, wherein step i) further comprises the step of collimating the electromagnetic radiation emitted by the radiopharmaceutical prior to the reception by the one or more radiation sensors.

5. An apparatus for the non-invasive and extra-corporally determination of the arterial concentration of a radiopharmaceutical, in particular a radiotracer administered in an organ of an animal and/or human body, the apparatus comprising:
one or more radiation sensors to be positioned near or on a measurement location of an organ of the animal and/or human body and configured to receive electromagnetic radiation emitted by the radiopharmaceutical over time;
a conversion unit configured to convert the received electromagnetic radiation over time in a time-sequence of radiation signals;
at least one processing unit configured to generate one or multiple time-frequency representations of the time-sequence of radiation signals through time-frequency transformation, and for determining the arterial concentration of the radiopharmaceutical.

6. The apparatus of claim 5, further comprising an output unit configured to output the concentration of the radiopharmaceutical being determined.

7. The apparatus of claim 5, wherein the time-frequency transformation is selected from the group exemplified by but not limited to short-time Fourier transform, wavelet transform, filter bank, or discrete cosine transform.

8. The apparatus of claim 5, wherein two or more radiation sensors are positioned at opposite sides of the measurement location of the organ.

9. The apparatus of claim 8, further comprising a coincidence processing unit structured to detect the simultaneous detection of electromagnetic radiation by the two or more radiation sensors positioned at opposite sides of the measurement location of the organ.

10. The apparatus of claim 5, further comprising a housing for accommodating at least the one or more radiation sensors, as well as radiation collimating means positioned around each of the one or more radiation sensors.

11. The apparatus of claim 5, wherein the apparatus is formed as a wearable device, for example structured for wearing around a limb of the animal and/or human body.

12. The apparatus of claim 5, wherein the apparatus is structured to positioned near, at or incorporated in a hospital chair, hospital bed or patient table.

13. A computer program or product comprising instructions which, when the program is executed by a computer, cause the computer to carry out the steps of the computer implemented method of claim 1.

14. A computer-readable storage medium comprising instructions which, when executed by a computer, cause the computer to carry out the steps of the computer implemented method of claim 1.
